(19) United States
(12) Patent Application Publication
(10) Pub. No.: US 2005/0228354 A1
(43) Pub. Date: Oct. 13, 2005

(54) Resealable diaper package

(76) Inventor: Joelle J. Scholer, Cincinnati, OH (US)

Correspondence Address: WOOD, HERRON & EVANS, LLP, 2700 CAREW TOWER, 441 VINE STREET, CINCINNATI, OH, 45202, US

(21) Appl. No.: 11/100,769

(22) Filed: Apr. 7, 2005

Related U.S. Application Data
(60) Provisional application No. 60/560,229, filed on Apr. 7, 2004.

(51) Int. Cl. A61F 13/15

(52) U.S. Cl. 604/385.02; 604/385.13

(57) ABSTRACT

A resealable diaper bag keeps a diaper clean until the diaper is used. The resealable diaper bag then receives and encloses a soiled diaper for disposal.

### RELATED APPLICATIONS

This application claims the benefit of U.S. Provisional Application No. 60/560,229 filed on Apr. 7, 2004, the entire disclosure of which is hereby incorporated by reference herein.

### FIELD OF THE INVENTION

The present invention relates to diapers, and more particularly, to the disposal of diapers.

### BACKGROUND OF THE INVENTION

Few things stink like a dirty diaper. Yet the unpleasant odor of soiled diapers is only a part of what makes their disposal universally unpalatable. To this end, efforts have been made to improve diaper disposal processes. For instance, disposable diapers and wet wipes largely obviated the need for washing diapers. Furthermore, disposable diapers are conventionally constructed such that they fold in on themselves to ideally contain their soiled contents. For example, hook and fastener straps that are initially used to secure the diaper to a baby are used after the diaper is soiled to hold the diaper in the folded, contained state. Despite achieving some odor and excrement containment, diaper contents may still escape from a folded disposable diaper.

Largely for this reason, diaper bins have been developed to receive and temporarily store soiled disposable diapers. Such bins may include a canister having a plastic interior bag configured to receive a diaper. A lid fitted to the top of the cannister may be turned or otherwise manipulated to seal a soiled, deposited diaper within the plastic bag of the diaper bin. Such diaper bins help contain unpleasant smells, however, they are not always available to users. For example, diaper bins are configured to store many soiled diapers and are consequently relatively bulky. As such, most parents cannot travel conveniently with a diaper bin. The unavailability and impracticality of the diaper bin often relegates parents to disposing of diapers wherever and however possible. Even where available, unpleasant smells from the contents of the diaper bins still escape.

Diaper kits have been developed that include a diaper and an attached wipe. The wipe is initially stored inside a package. The wipe package may later be used to hold a soiled diaper. These diaper kits can be expensive to manufacture and package. Additionally, more than one wipe is sometimes needed to change a diaper. The diaper, itself, may remain unprotected from corrupting elements, such as dirt, leaking bottles, etc. Other diaper kits require the user to tear off portions of a bag to access a contained diaper, resulting in wasted bag material and potential frustration for a hurried care giver. Consequently, an improved mechanism is needed for disposing of soiled diapers.

### SUMMARY OF THE INVENTION

This invention provides an improved apparatus and method for disposing of a soiled diaper. More particularly, a resealable diaper bag encloses a new diaper such that a user can remove the new diaper from the resealable bag. Once the fresh diaper is removed from the resealable bag, the resealable bag may be used to enclose and seal within the bag at least one soiled diaper. The resealable diaper package of the present invention includes a diaper and a resealable bag configured to temporarily enclose the diaper. The resealable bag includes first and second opposing surfaces configured to form a temporary seal when contacted with each other, and to form an opening through which the diaper is removed from the resealable bag when not contacted. One or both of the first and second surfaces includes a fastener used to form the temporary seal, and the resealable bag is further configured to enclose using the fastener at least one soiled diaper after the diaper has been removed from the resealable bag.

The mechanism that allows the bag to be resealed may include a fastener. Exemplary fasteners may include hook-and-eye fasteners, adhesive, cling-wrap material, a metal-twist, a rubber band, a snap, an eye-loop fastener, a hook, a zipper, string and a Ziplock® fastener. The bag, or package may be constructed of any combination of plastic, mesh, cloth, polymer, metal or glass.

The dimensions of the resealable bag may be configured according to the absorbency and/or the expected load of a soiled diaper. That is, the dimensions may be made large enough to contain and seal a soiled diaper, which will have a larger volume than the fresh diaper originally contained within the bag. For instance, a used diaper can have at least twice the volume as a fresh diaper due to its liquid and/or solid contents.

Diaper bags in accordance with the principles of the present invention may additionally be sized and proportioned to accommodate a soiled diaper that has been folded in on itself. For instance, the bag may be configured such that it can hold more in its middle than at its end, for instance, to better accommodate the shape of a wadded, used diaper. Where desired, the fresh diaper may be folded or otherwise postured inside of the bag prior to use to accommodate the shape of the bag. In any case, an embodiment of the present invention may actually complement conventional techniques and improvements.

In another embodiment, the dimensions of the bag are configured according to the stretchability of the material used to make the bag. For instance, a resealable bag that is consistent with the principles of the present invention may have dimensions only slightly larger than a fresh diaper, and the bag may later be stretched to accommodate a relatively larger, soiled diaper. In any case, combinations of sizing and material features of the bag may both be used when configuring a resealable package.

By virtue of providing a resealable diaper bag, smells and diaper contents may be better contained. For instance, diapers may be resealed and disposed of as any with any other lifter without the conventional stigma and odor associated with conventional diaper disposal. Such a feature may alleviate problems conventionally associated with traveling with babies. Where desired, resealed diapers may be disposed of within conventional diaper bins for further containment. In this sense, another embodiment of the present invention further complements conventional techniques and improvements.

By virtue of the foregoing there is thus provided an improved diaper disposal system that addresses shortcomings of conventional techniques. These and other objects and advantages of the present invention shall be made apparent in the accompanying drawings and the description thereof.

### BRIEF DESCRIPTION OF THE DRAWINGS

The accompanying drawings, which are incorporated in and constitute a part of this specification, illustrate an embodiment of the invention and, together with a general description of the invention given above, and the detailed description of the embodiment given below, serve to explain the principles of the invention.

FIG. 1 shows a diaper removed from a resealable diaper package that is consistent with the principles of the present invention.

FIG. 2 shows the resealable diaper package of FIG. 1 containing a soiled diaper.

FIG. 3 shows a resealable diaper package containing a diaper in a manner that is consistent with the principles of the present invention.

FIG. 4 shows a cross-sectional view of a resealable diaper bag that is consistent with the principles of the present invention.

### DETAILED DESCRIPTION OF THE INVENTION

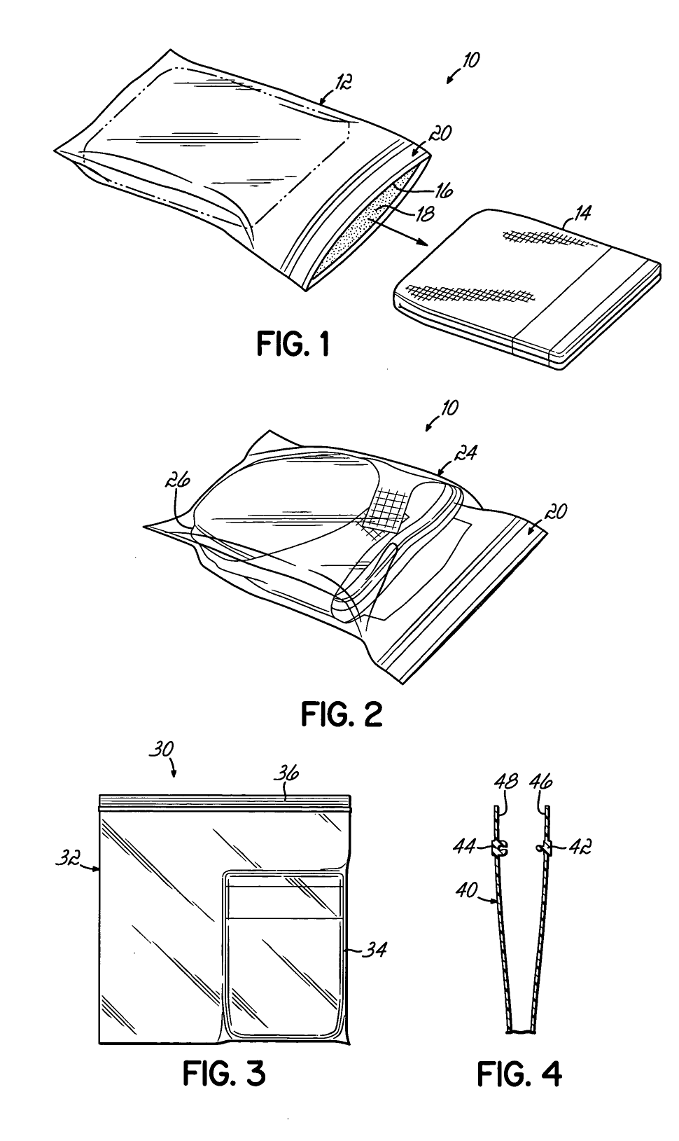

FIG. 1 shows a diaper package system 10 that is consistent with the principles of the present invention. The system includes a resealable diaper bag, or package 12. A fresh diaper 14 is shown in FIG. 1, shown removed from the diaper package 12. The diaper package 12 is shown in FIG. 1 as containing only one diaper 14. However, one skilled in the art will appreciate that other packages consistent with the present invention may include other numbers of diapers, e.g., about two to ten.

The diaper package 12 includes first and second surfaces 16, 18. As shown, surface 16 includes a resealable fastener 20. Such a fastener 20 may comprise an adhesive, for instance. The fastener is typically, but not necessarily, the same fastener that originally seals the diaper package 12. The user may then reapply the adhesive side(s) of the surfaces 16, 18 together to make them reseal the package 12 after a dirty diaper has been placed in the package 12. Other fasteners may include tape, hook-and-eye, an adhesive under a peel-off strip, cling-wrap, metal twist, rubber band, snaps, eye-loop fasteners, hook, zipper, string and Ziplock® features. Typically, the opposing sides 16, 18 are not damaged when the diaper 14 is removed, so that the opposing sides 16, 18 can form the temporary seal without wasting material used to construct the package.

The diaper package 12, itself, may comprise any combination of at least one of: plastic, mesh, cloth, polymer, metal and glass. The diaper package 12 may be sized according to expected dimensions and/or absorbency of a soiled diaper. Other considerations of the dimensions of the bag 12 may include the stretchability or give of the material used to construct the bag 12. Thus, the package 12 may be oversized and/or configured to stretch and accommodate a soiled diaper. More than one fastening/opening section of the bag may be used where more convenient. The soiled diaper sealed in the resealable package may or may not be the fresh diaper originally removed from the package 12. One skilled in the art will appreciate that the diaper package 12 need not be rectangular shaped or even shaped to fit the shape of a fresh diaper. The resealable package 12 may be scented and/or decorated where desired.

FIG. 2 shows a resealable diaper package system 10, like that shown in FIG. 1, except the package 24 that has been sealed to contain a soiled diaper 26. As shown in FIG. 2, the package 24 has conformed to the shape of the folded-in, wadded up dirty diaper 26.

FIG. 3 shows an unopened, resealable diaper package system 30 that includes a package 32 containing a fresh diaper 34 and a fastener 36. The diaper package 32 is shown as oversized compared to the diaper 34. Where desired, the excess area of the package 32 may be been vacuum aired out to decrease the volume of the bag for packaging considerations. One skilled in the art will appreciate that the package may be folded in on itself during packaging to further decrease volume, where desired. Such a feature may facilitate packaging in bulk. In this vein, diapers contained in their respective resealable bags may be stacked and packaged within bulk packages in much the same way that are conventional disposable diapers. That is, diapers may be packaged in the resealable packages as discussed herein prior to transport, storage and sale.

FIG. 4 shows a cross-sectional view of a resealable diaper bag 40 that is consistent with the principles of the present invention. The bag 40 includes a fastener comprising male and female interlocking components 42, 44, positioned along opposing surfaces 46, 48 of the bag 40.

Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of the applicants' general inventive concept.

1. An apparatus comprising:
a diaper; and
a resealable bag configured to temporarily enclose the diaper, the resealable bag including first and second opposing surfaces configured to form a temporary seal when contacted with each other, and to form an opening through which the diaper is removed from the resealable bag when not contacted, wherein at least one of the first and second surfaces includes a fastener used to form the temporary seal, and wherein the resealable bag is further configured to enclose using the fastener at least one soiled diaper after the diaper has been removed from the resealable bag.
